(19) United States
(12) Patent Application Publication
(10) Pub. No.: US 2006/0234277 A1
(43) Pub. Date: Oct. 19, 2006

(54) Method for selectively blocking hemoglobin RNA amplification

(75) Inventors: Chris B. Russell, Bainbridge Island, WA (US); Keith Kerkof, Seattle, WA (US); Martin Timour, Issaquah, WA (US)

Correspondence Address: AMGEN INC.;LAW DEPARTMENT, 1201 AMGEN COURT WEST, SEATTLE, WA, 98119, US

(73) Assignee: Amgen Inc., Thousand Oaks, CA (US)

(21) Appl. No.: 11/396,037

(22) Filed: Mar. 31, 2006

Related U.S. Application Data
(60) Provisional application No. 60/667,488, filed on Mar. 31, 2005.

(51) Int. Cl. C12Q 1/68; C07H 21/02; C12P 19/34

(52) U.S. Cl. 435/6; 435/91.2; 536/23.1

(57) ABSTRACT

This invention provides oligonucleotides, compositions, kits, and methods for specifically blocking amplification of hemoglobin mRNA in a sample of RNA. The oligonucleotides and methods are particularly advantageous for analyzing samples of RNA extracted from whole blood samples.

This application hereby claims benefit of U.S. provisional application Ser. No. 60/667,488, filed Mar. 31, 2005, the entire disclosure of which is relied upon and incorporated by reference.

### FIELD OF THE INVENTION

This invention relates to specific oligonucleotides, compositions, kits and methods for blocking amplification of selected RNA transcripts.

### BACKGROUND OF THE INVENTION

A number of methods for selectively suppressing amplification of selected polynucleotides have been proposed. Blocking amplification of undesired targets has a number of advantages. One advantage is the ability to detect rarer sequences in a sample containing a large number of highly abundant sequences. One example of selective amplification was described in Orum et al, Nucleic Acids Res 21 (23), 5332-5336 (1993). Orum et al. described the technique of peptide nucleic acid (PNA) directed polymerase chain reaction (PCR) “clamping”. This technique involves generating sequences containing PNAs directed against primer sites on the undesired target, thereby blocking PCR product formation. This is due to the fact that a PNA/DNA complex is more heat stable than a corresponding DNA/DNA duplex, and additionally, PNA cannot function as a primer for DNA polymerases. Therefore PNA can block PCR amplification in a sequence specific manner. This technique was used to distinguish single base mutations in specific genes by blocking amplification of sequences which differ by only one base pair from the desired sequence.

A second technique for blocking unwanted targets was described in Seyama et al. Nucleic Acids Res 20 (10), 2493-2496 (1993). This technique was also used to selectively amplify single base pair mutations over normal alleles. This technique relies on the use of a complementary pair of oligonucleotides located between the two primers, having the 3′ ends labeled with dideoxynucleotides to prohibit their elongation by DNA polymerase. Under normal annealing conditions, the blockers hybridize to normal alleles more preferentially than to mutated alleles bearing base mutations because of the presence of the mismatched base pair in the blocker mutant hybrids. As a consequence, DNA replication of the mutant alleles proceeds preferentially compared to DNA replication of the normal allele, and selective amplification is achieved.

Blocking amplification of highly abundant sequences in order to better detect rarer sequences is particularly useful when individuals or populations are being screened for expression of genes associated with a disease state, or associated with a pharmacological response to a particular therapeutic treatment. Tissue samples are collected from individual patients or populations of individuals for comparison to each other, or for screening over time, for example. The samples can be monitored for messenger ribonucleic acid (mRNA) levels. mRNA can be amplified and then detected using a number of technologies. Particular transcripts can be detected or monitored by electrophoresis, for example. More commonly, large numbers of transcripts correlated to gene expression are monitored using expression arrays, which contain embedded probes to which labeled transcripts present in the sample hybridize on the surface of a chip. Changes in the expression patterns can then be detected by microarray analysis.

When the samples collected for analysis are whole blood samples, particular problems with detecting rarer sequences are encountered due to the predominance of hemoglobin RNA in RNA samples extracted from whole blood. The present invention provides oligonucleotides, compositions, kits and oligonucleotides for overcoming these problems by selectively blocking the amplification of hemoglobin in the whole blood samples.

### SUMMARY OF THE INVENTION

The present invention provides oligonucleotides, compositions, methods and kits for blocking amplification of hemoglobin messenger ribonucleic acid (mRNA) transcripts present in an RNA sample. The oligonucleotides of the present invention act to block amplification of hemoglobin A1 (HBA1), hemoglobin A2 (HBA2), or hemoglobin B (HBB) mRNA transcripts or combinations of these by hybridizing to the 3′ terminal of the transcript. In one embodiment, the oligonucleotides of the present invention have between about 8 and about 30 total nucleotides, in another embodiment, between about 8 and about 20 nucleotides, and in another embodiment, between about 10 and about 18 nucleotides. The oligonucleotides of the present invention comprise at least one modified nucleotide analog having a locked structure.

The oligonucleotides of the present invention act to form a heat stable duplex with the 3′ terminal of the hemoglobin transcript being targeted, preventing amplification of the targeted transcript. In one embodiment, the Tm of these duplexes is between about 58° C. and about 84° C., in another embodiment, between about 60° C. and about 82° C.

In one embodiment the oligonucleotides of the present invention are selected from the following sequences: GCCCACtcacAGA (SEQ ID NO: 1), CCCTTcataatatCCC (SEQ ID NO: 2), TTGccgcccACTC (SEQ ID NO: 3), CAAtgAAAAtAAATG (SEQ ID NO: 4), TTGccgcccACTCA (SEQ ID NO: 5), and TTTAttcaaagaCCA (SEQ ID NO: 6), wherein the capital letters represent modified locked nucleotide analogs, and the small letters represent conventional deoxyribonucleotides. These oligonucleotides can be used individually or in combination to suppress amplification of hemoglobin mRNA transcripts. The oligonucleotides of the present invention further includes oligonucleotides with one or more substitutions to the sequences listed above, wherein locked nucleotides may be substituted for conventional nucleotides, and conventional nucleotides may be substituted for locked nucleotides, provided that the Tm of the resulting oligonucleotide maintains the approximate Tm of the original oligonucleotide.

The present invention further provides compositions and kits for use in preparing whole blood samples for amplification comprising the oligonucleotides of the present invention.

The present invention also provides methods for blocking the amplification of hemoglobin mRNA transcripts by treating an RNA sample containing hemoglobin mRNA with one or more of the olignonucleotides of the present invention. In one embodiment, the oligonucleotides are used pretreat an RNA sample prior to the addition of amplification enzymes to the sample. The use of the oligonucleotides of the present invention to specifically suppress hemoglobin amplification allows for the amplification and detection of non-hemoglobin transcripts present in a biological sample. The present invention is particular advantageous for use in analyzing RNA samples derived from whole blood to identify variations in non-hemoglobin gene expression.

### BRIEF DESCRIPTION OF THE FIGURES

FIG. 1 shows the structure of one embodiment of a locked nucleotide analog monomer in comparison to an RNA monomer.

FIG. 2 shows a comparison between a sample of MRNA treated with oligonucleotides #2 and #3, and an identical sample which was not treated with these oligonucleotides. The labeled cRNA final products were analyzed on an Agilent 2100 bioanalyzer. The upper line shows the labeled hemoglobin peak from the untreated sample, while the lower line shows the pretreated RNA sample in which hemoglobin amplification and subsequent labelling has been blocked.

FIG. 3 show a comparison of signals generated from GeneChip® (Affymetrix) analysis. FIG. 3A shows a comparison between two different samples of labelled RNA pretreated with oligos and not pretreated. FIG. 3B shows a comparison between two different samples of labelled RNA treated with a different hemoglobin reduction protocol (globin reduction protocol using RNAse) and not treated. This comparison shows that the oligo pretreatment of the present invention produces more consistent and reproducible results between samples by reduced labelling of non-hemoglobin targets.

### DETAILED DESCRIPTION OF THE INVENTION

The present invention provides specific oligonucleotides designed to directly block amplification of hemoglobin mRNA transcripts. These oligonucleotides hybridize with the 3′ end of one or more hemoglobin mRNA transcripts, forming a thermostable duplex capable of blocking amplification of the targeted transcripts.

Hemoglobin mRNA

The oligonucleotides and methods of the present invention are designed to block hemoglobin mRNA amplification and labelling. This method is especially useful for analyzing RNA samples taken from whole blood.

It is desirable to be able to analyze changes in gene expression in human subjects due to disease state, or in response to therapeutic treatments. A number of technologies such as expression microarray analysis have been developed for gene expression analysis. One way this can be accomplished is by amplifying and labelling RNA taken from tissue samples, and detecting transcripts capable of hybridizing to nucleic acid sequences on the chips. Whole blood samples are the most easily and conveniently obtaining from living human subjects. Whole blood contains erythrocytes or red blood cells, and leukocytes or white blood cells. Red blood cells contain hemoglobin is a tetrameric molecule that carries oxygen throughout the circulatory system. Approximately 70 to 80% of the messenger RNA in whole blood is hemoglobin mRNA. These transcripts can overwhelm and obscure the detection of less frequently expressed transcripts in RNA obtained from whole blood samples. When labelling RNA for hybridization to DNA chips, for example, the large number of hemoglobin transcripts dominates the labelling reaction, saturates the hemoglobin probe sets, and cross-hybridizes to many other sequences. The present invention provides particular sequences and methods for blocking amplification of undesired hemoglobin RNA transcripts, thereby allowing detection of less frequently expressed RNA transcripts present in whole blood samples.

Hemoglobin is a tetrameric protein made up of two alpha and two beta subunits. The human alpha globin gene cluster is located on chromosome 16 and spans about 30 kb and includes the following five loci: 5′-zeta-pseudozeta-pseudoalpha-1-alpha-2 -alpha-1-3′. The alpha-1 (HBA1) and alpha-2 (HBA2) coding sequences are identical. Collectively, HBA1 and HBA2 are referred to as HBA. These genes differ slightly over the 5′ untranslated regions and the introns, and they differ significantly over the 3′ untranslated regions. The human beta globin gene (HBB) codes for the beta hemoglobin subunit and is located at another locus. The three hemoglobin sequences are provided in Table 1.

TABLE 1
HEMOGLOBIN SEQUENCES

| | | | |
|---|---|---|---|
| HBA1 | 1 | actcttctgg tccccacaga | (SEQ ID NO:7) |
| Accession No. | | ctcagagaga acccaccatg | |
| NM_000558.3 | | gtgctgtctc | |
| | 51 | ctgccgacaa gaccaacgtc | |
| | | aaggccgcct ggggtaaggt | |
| | | cggcgcgcac | |
| | 101 | gctggcgagt atggtgcgga | |
| | | ggccctggag aggatgttcc | |
| | | tgtccttccc | |
| | 151 | caccaccaag acctacttcc | |
| | | cgcacttcga cctgagccac | |
| | | ggctctgccc | |
| | 201 | aggttaaggg ccacggcaag | |
| | | aaggtggccg acgcgctgac | |
| | | caacgccgtg | |
| | 251 | gcgcacgtgg acgacatgcc | |
| | | caacgcgctg tccgccctga | |
| | | gcgacctgca | |
| | 301 | cgcgcacaag cttcgggtgg | |
| | | acccggtcaa cttcaagctc | |
| | | ctaagccact | |
| | 351 | gcctgctggt gaccctggcc | |
| | | gcccacctcc ccgccgagtt | |
| | | cacccctgcg | |
| | 401 | gtgcacgcct ccctggacaa | |
| | | gttcctggct tctgtgagca | |
| | | ccgtgctgac | |
| | 451 | ctccaaatac cgttaagctg | |
| | | gagcctcggt ggccatgctt | |
| | | cttgcccctt | |
| | 501 | gggcctcccc ccagcccctc | |
| | | ctccccttcc tgcacccgta | |
| | | cccccgtggt | |
| | 551 | ctttgaataa agtctgagtg | |
| | | ggcggc | |
| HBA2 | 1 | actcttctgg tccccacaga | (SEQ ID NO:8) |
| Accession No. | | ctcagagaga acccaccatg | |
| NM_000517.3 | | gtgctgtctc | |
| | 51 | ctgccgacaa gaccaacgtc | |
| | | aaggccgcct ggggtaaggt | |
| | | cggcgcgcac | |
| | 101 | gctggcgagt atggtgcgga | |
| | | ggccctggag aggatgttcc | |
| | | tgtccttccc | |
| | 151 | caccaccaag acctacftcc | |
| | | cgcacttcga cctgagccac | |
| | | ggctctgccc | |
| | 201 | aggttaaggg ccacggcaag | |
| | | aaggtggccg acgcgctgac | |
| | | caacgccgtg | |
| | 251 | gcgcacgtgg acgacatgcc | |
| | | caacgcgctg tccgccctga | |
| | | gcgacctgca | |
| | 301 | cgcgcacaag cttcgggtgg | |
| | | acccggtcaa cttcaagctc | |
| | | ctaagccact | |
| | 351 | gcctgctggt gaccctggcc | |
| | | gcccacctcc ccgccgagtt | |
| | | cacccctgcg | |
| | 401 | gtgcacgcct ccctggacaa | |
| | | gttcctggct tctgtgagca | |
| | | ccgtgctgac | |
| | 451 | ctccaaatac cgttaagctg | |
| | | gagcctcggt agccgttcct | |
| | | cctgcccgct | |
| | 501 | gggcctccca acgggccctc | |
| | | ctcccctcct tgcaccggcc | |
| | | cttcctggtc | |
| | 551 | tttgaataaa gtctgagtgg | |
| | | gcggc | |
| HBB | 1 | acatttgctt ctgacacaac | (SEQ ID NO:9) |
| Accession No. | | tgtgttcact agcaacctca | |
| NM_000518.4 | | aacagacacc | |
| | 51 | atggtgcatc tgactcctga | |
| | | ggagaagtct gccgttactg | |
| | | ccctgtgggg | |
| | 101 | caaggtgaac gtggatgaag | |
| | | ttggtggtga ggccctgggc | |
| | | aggctgctgg | |
| | 151 | tggtctaccc ttggacccag | |
| | | aggttctttg agtcctttgg | |
| | | ggatctgtcc | |
| | 201 | actcctgatg ctgttatggg | |
| | | caaccctaag gtgaaggctc | |
| | | atggcaagaa | |
| | 251 | agtgctcggt gcctttagtg | |
| | | atggcctggc tcacctggac | |
| | | aacctcaagg | |
| | 301 | gcacctttgc cacactgagt | |
| | | gagctgcact gtgacaagct | |
| | | gcacgtggat | |
| | 351 | cctgagaact tcaggctcct | |
| | | gggcaacgtg ctggtctgtg | |
| | | tgctggccca | |
| | 401 | tcactttggc aaagaattca | |
| | | ccccaccagt gcaggctgcc | |
| | | tatcagaaag | |
| | 451 | tggtggctgg tgtggctaat | |
| | | gccctggccc acaagtatca | |
| | | ctaagctcgc | |
| | 501 | tttcttgctg tccaatttct | |
| | | attaaaggtt cctttgttcc | |
| | | ctaagtccaa | |
| | 551 | ctactaaact gggggatatt | |
| | | atgaagggcc ttgagcatct | |
| | | ggattctgcc | |
| | 601 | taataaaaaa catttatttt | |
| | | cattgc | |

Oligonucleotides

The oligonucleotides (also referred to as “oligos” or “oligomers”) of the present invention contain at least one modified nucleotide analog in combination with naturally occurring conventional nucleotides. The oligonucleotides of the present invention are between about 8 and 30 nucleotides in length in one embodiment, between about 8 and 20 nucleotides in length in another embodiment, and between about 10 and 18 nucleotides in length in another embodiment.

The oligonucleotides are designed to hybridize with the 3′ terminal of one or more hemoglobin transcripts, with minimal cross-hybridization to other potential targets. More specifically, the oligonucleotides of the present invention are designed to hybridize to both hemoglobin RNA transcripts HBA-1 and HBA-2 (collectively called HBA), or to HBB. The oligonucleotides hybridize to the 3′ terminal of the HBA or HBB transcripts, preventing amplification of the targeted transcripts by forming a thermostable duplex. The duplex is thought to present a poor substrate for enzymes required for amplification of cDNA.

As used herein the term “nucleoside”, “conventional nucleoside” refers to the ribonucleoside and deoxyribonucleoside monomers adenosine, guanosine, cytidine, uridine, thymidine, their deoxy counterparts, and other naturally occurring monomers of DNA (deoxyribonucleic acid) and RNA (ribonucleic acid). Nucleosides are a nitrogenous base, purine (adenine and guanine) or pyrimidine (cytosine, uracil, thymine) linked to the C1′ of a pentose sugar (ribose for RNA and deoxyribose for DNA). Nucleotides are the phosphate esters of the nucleoside. When included within a larger sequence, the term “nucleotide” is used for an RNA or DNA monomer. The oligonucleotides of the present invention contain a mixture of both conventional nucleotides and nucleotide analogs.

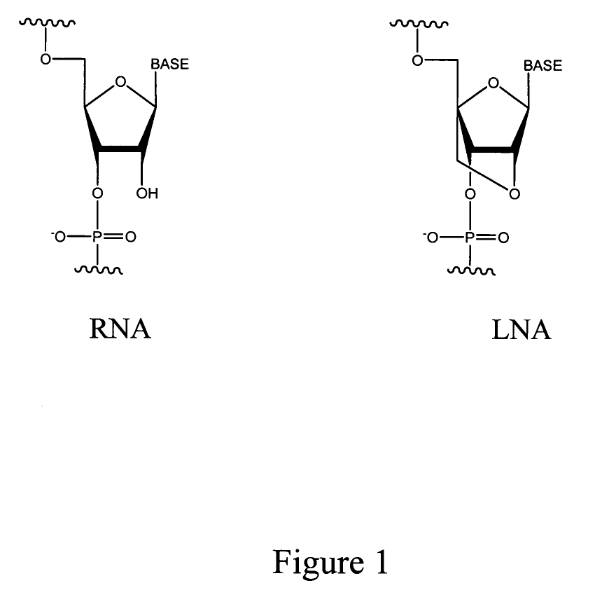

As used herein, the term “analog” generally refers to a modified nucleoside or nucleotide monomer. When the monomer is included within a larger sequence, the analog is a modified nucleotide. As used herein, the term “locked nucleoside analog” or “locked nucleotide analog”, referred to as “LNA” or “LNA monomer”, refers to an nucleoside or nucleotide modified to contain a “locked” structure. This class of analogs are described in U.S. Pat. Nos. 6,749,499; 6,734,291; and 6,670,461, all of which are incorporated by reference herein, and in Koshkin et al, Tetrahedron 54: 3607-3630 (1998). In one embodiment, locked nucleoside or nucleotide analogs are bi- or tricyclic nucleosides or nucleotides that are analogous to DNA or RNA nucleoside or nucleotide monomers but contain a locked structure. In one embodiment, the locked structure is a 2′-O, 4′-C methylene bridge of the sugar, as shown in FIG. 1. As used herein the term “locked nucleic acid” refers to an oligonucleotide or polynucleotide containing at least one locked nucleotide analog monomer.

The base substituent of an LNA monomer may be selected from known purines and pyrimidines, as well as heterocyclic analogs and tautomers thereof. Examples of bases include both naturally occuring and non-naturally occuring bases including but not limited to adenine, guanine, thymine, cytosine and uracil, as well as purine, xanthine, diaminopurine, 8-oxo-N⁶ -methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-5-methyl-4-triazolpyridin, isocytosine, isoguanine, inosine and other non-naturally occurring nucleobases as described for example in U.S. Pat. 6,670,461, and U.S. Pat. No. 5,432,272, which are herein incorporated by reference. In addition, the LNA monomers may be made with substituents other than the bases described above, wherein the substituent is a group capable of interacting via hydrogen or covalent bonding with the bases of DNA or RNA. These substituents may include hydrogen, hydroxyl, C₁₋₄-alkoxy, C₁₋₄-alkyl, C,₁₋₄-acyloxy, DNA intercalators, photochemically active groups, thermochemically active groups, chelating groups, reporter groups, and ligands as described for example, in U.S. Pat. No. 6,670,461.

The chemical synthesis of LNA monomers is described in detail in Koshkin et al., supra, at 3609. In one embodiment, a 4′-C-hydroxymethly pentofuranose deriviative (described in Youssefyeh et al, J Org. Chem 44, 1301 (1979)) was chosen as a starting material. 5-O-benzylation, acetylation, and acetolysis followed by acetylation produced the intermediate furanose, to which can be coupled a variety of silylated nucleobases (Koshkin et al, 3609). Locked analogs of the various convention nucleosides can be produced in this manner. LNA monomers are also available commercially as LNA® phophoramidites (Proligo Reagents™, LLC, Boulder, Colo.), which can be incorporated into a particular oligonucleotide using the vendor's instructions. LNA phosphoramidites can be used to generate oligonucleotides containing a mixture of nucleotide analogs and conventional nucleotides.

The oligonucleotides of the present invention include at least one LNA monomer, in one embodiment, between about two and about fifteen LNA monomers, in another embodiment between about seven and fifteen LNA monomers, in combination with conventional nucleotides. The placement of the analogs in the oligonucleotide sequence is designed to achieve a high affinity and specificity for the hemoglobin transcript it is targeting. The high thermal stability of the duplex formed by the oligonucleotides and its targeted hemoglobin transcript prevents subsequent amplification of that transcript. The high specificity of the oligonucleotides is designed to prevent cross-hybridization of the oligos with other unintended targets. Low cross-hybridization of non-hemoglobin targets allows for consistent, reproducible results between different samples.

Oligonucleotides containing LNA monomers may be synthesized using the phosphoramidite approach, as described in Caruthers et al. Acc.Chem Res 24, 278 (1991), using the same reagents used for DNA synthesis. For example, standard coupling conditions using DNA synthesizers (such as Pharmacia Gene Assembler Special®. Biosearch 8750 DNA Synthesizer) can be used, except that the coupling time for LNA amidites is increased compared with conventional nucleosides. Standard 2′deoxynucleoside CPG or polystyrene solid supports can be used, or a Universal CPG Support (BioGenex) can be used (Koshkin et al. supra, at 3611) to produce the oligonucleotides. After completion of desired synthetic sequences of steps including final cleavage and deprotection, the oligonucleotides can be purified using chromotography such as reverse phase chromotography (Koshkin et al, supra at 3611). Additional synthetic methods include various deprotection chemistries can be used in conventional automated phosphoramidite oligonucleotide synthesis.

Synthesis of the oligonucleotides containing LNA monomers can also be performed using commercially available synthesis columns (Proligo Reagents™, Proligo LLC) according to manufacturer's instructions. Alternatively, oligonucleotides can be specifically designed by the user and produced commercially according to the user specification (Proligo, LLC).

Specific Oligonucleotides

The oligonucleotides of the present invention are selected from the following sequences: GCCCACtcacAGA (SEQ ID NO: 1), CCCTTcataatatCCC (SEQ ID NO: 2), TTGccgcccACTC (SEQ ID NO: 3), CAAtgAAAAtAAATG (SEQ ID NO: 4), TTGccgcccACTCA (SEQ ID NO: 5), and TTTAttcaaagaCCA (SEQ ID NO: 6). The capital letters in the sequence represents the locked nucleotide analog containing a 2′-O, 4′-C-methylene bridge, while the small letters refer to the conventional deoxyribonucleotides. These oligonucleotides can be used individually or in combination to block amplification of hemoglobin mRNA transcripts.

The oligonuleotides designed to hybridize and form a thermostable complex with the 3′ end of both HBA1 and HBA2 are the following: GCCCACtcacAGA (SEQ ID NO: 1); TTGccgcccACTC (SEQ ID NO: 3); TTGccgcccACTCA (SEQ ID NO: 5); and TTTAttcaaagaCCA (SEQ ID NO: 6).

A second group of oligonucleotides designed to hybridize with and form a thermostable complex with the 3′ end of the HBB mRNA transcript are the following:

| | |
|---|---|
| CCCTTcataatatCCC, | (SEQ ID NO:2) |
| and | |
| CAAtgAAAAtAAATG. | (SEQ ID NO:4) |

Locked nucleotide analogs incorporated into oligonucleotides confers specific properties on the oligonucleotides containing them. The properties are determined by the number and placement of the LNA contained in the oligonucleotides. The locked conformation of the nucleotide analogs affects the adjacent nucleotides in the oligonucleotides, conferring increased stability and increased melting temperatures on the duplexes formed. The oligonucleotides of the present invention form duplexes with complementary RNA or DNA sequences which are more thermally stable than duplexes formed with complementary RNA or DNA oligos. LNA:RNA or LNA:DNA duplexes have a higher Tm than an RNA:DNA, RNA:RNA or DNA:DNA duplex. The stable duplexes formed with specific RNA target sequences are thought to interfere with enzyme function such as nucleases and polymerases, including reverse transcriptase.

The oligonucleotides of the present invention further includes oligonucleotides with one or more substitutions to the sequences listed above, wherein locked nucleotides may be substituted for conventional nucleotides, and conventional nucleotides may be substituted for locked nucleotides, provided that the Tm of the resulting oligonucleotide maintains the approximate Tm of the original oligonucleotide.

The oligonucleotides were designed to have an approximate Tm which allows for specificity in binding to the desired hemoglobin targets and reduction in cross-hybridization with unintended targets. The Tm is determined for a specific sequence of locked and conventional nucleotides using parameters described in Tolstrup et al., Nuc Acid Res 31:3758-3762 (2003). The Tm range of the oligonucleotides of the present invention varies from about 58° C. to about 84° C. in one embodiment, and between about 60° C. to about 82° C. in another embodiment. The Tm of the specific sequences 1 to 6 are given below.

| | | | |
|---|---|---|---|
| HBA oligo #1 | (GCCCACtcacAGA) | 78° C. | SEQ ID NO:1 |
| HBB oligo #2 | (CCCTTcataatatCCC) | 70° C. | SEQ ID NO:2 |
| HBA oligo #3 | (TTGccgcccACTC) | 78° C. | SEQ ID NO:3 |
| HBB oligo #4 | (CAAtgAAAAtAAATG) | 64° C. | SEQ ID NO:4 |
| HBA oligo #5 | (TTGccgcccACTCA) | 82° C. | SEQ ID NO:5 |
| HBA oligo #6 | (TTTAttcaaagaCCA) | 60° C. | SEQ ID NO:6 |

The present invention further provides compositions containing the oligonucleotides described above, as well as kits for treating RNA obtained from whole blood samples comprising the oligonucleotides described above.

Amplification

As used herein, the term “amplification” refers to a process for rapidly increasing the number targeted nucleic acid sequences to the level to which they can be detected. The most commonly used method is polymerase chain reaction or PCR. This method increases the numbers of specific sequences based on repeated cycles of denaturation of double-stranded polynucleotides, followed by annealing oligonucleotide primers to the single stranded polynucleotide templates, followed by primer extension using DNA polymerase. Methods of PCR have been described in U.S. Pat. Nos. 4,683,195, 4,683,202, and 4,800,159, which are herein incorporated by reference.

RNA samples may be amplified using a number of methods. For example, amplification can occur using reverse transcription to produce a first strand cDNA. As used herein the term “reverse transcription” refers to the replication of RNA using RNA-directed DNA polymerase (RT) to produce complementary strands of DNA (cDNA). First strand cDNA is synthesized from total RNA using oligo dT priming and a reverse transcriptase enzyme such as SuperScript II reverse transcriptase (Invitrogen, Carlsbad, Calif.). cDNA can then be further amplified using PCR amplification as described above by supplying specific primers to the PCR reaction mixture and running the mixture through a number of amplification cycles at specific temperatures to allow for denaturation, annealing and extension. For example temperatures in a thermocycler are alternated from a high temperature for denaturation, an intermediate temperature to allow for annealing, and a third temperature to allow for primer extension. As used herein, the term “amplification” with respect to RNA refers to processes which include the synthesis of cDNA.

Amplification of small amounts of RNA can also be performed using in vitro transcription (IVT) (Phillips et al. Methods 10, 283-288 (1996), Van Gelder et al., PNAS USA 87, 1663-1667 (1990), Baugh et al. Nucleic Acid Res 29 E29 (2001)). There are a number of in vitro amplification procedures known. One method of mRNA amplification by in vitro transcription of cDNA is based on a protocol first described by Eberwine et al. (Van Gelder et al, supra). In vitro amplification involves the addition of an RNA polymerase to a cDNA template along with ribonucleotides. This method may be used to produce cRNA from cDNA, which is useful in the preparation of labelled samples for microarray analysis.

The present invention further provides methods for specifically blocking hemoglobin RNA amplification in a sample of RNA by contacting the sample with one or more of the oligonucleotides of the present invention. In one embodiment, this method involves pretreating an RNA sample with the oligonucleotides of the present invention before contacting the sample with an enzyme such as an amplification enzyme. In one embodiment, one or more of oligonucleotides #1, #3, #5 or # 6 which bind to HBA are administered in combination with one or both of oligonucleotides #2 and #4 which bind to HBB. A specific exemplary protocol is provided in Example 2 below.

The present invention further provides a method of reducing the labelling of hemoglobin RNA in the preparation of whole blood RNA samples for further treatment and analysis. This is achieved by specifically blocking the amplification of hemoglobin RNA during the labelling process. Since seventy to eighty percent of blood mRNA is hemoglobin (HBA1, HBA2, HBB), hemoglobin mRNA dominates the labelling process, saturates the hemoglobin probe and can cross-hybridize with other sequences. The methods and oligonucleotides of the present invention are therefore particularly useful for preparing whole blood RNA samples for microarray analysis. Detailed descriptions of the practice of microarray labelling and analysis and the various commercially available microchips such as GeneChip® probe arrays (high density synthetic oligonucleotide arrays) are published, for example, Lipshutz et al, Nature Genet. 21, 21-24 (1999); Eisen et al, Methods Enzym 303, 179-205 (1999); Gerhold et al, Physiol. Genomics 5, 161-170 (2001); DeRisi et al., Science 278, 680-686 (1997); Shalon et al, Genome Res 6, 639-645 (1996); Kane et al, Nucl Acids Res 28,4552-4557 (2000); Rouillard et al, Bioinformatics 18, 486-487 (2002).

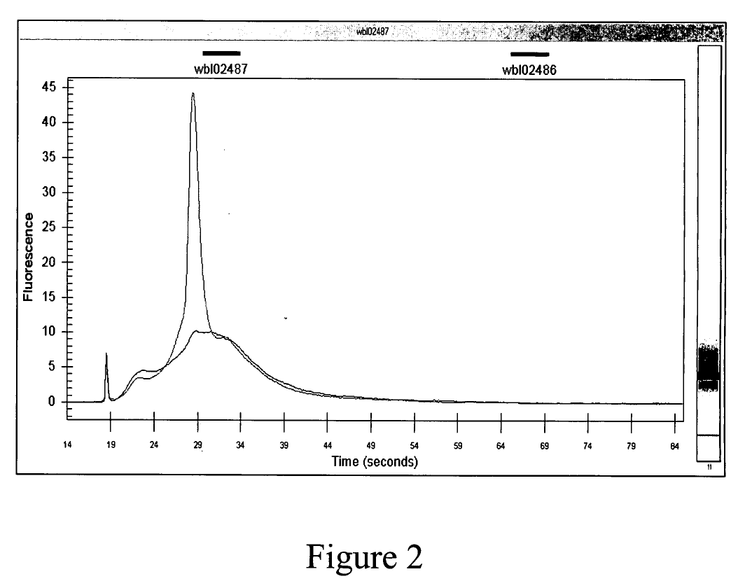

The following is a summary of an exemplary series of steps useful for labelling a sample of RNA derived from whole blood for microarray analysis. The oligonucleotides and methods of the present invention may be applied to additional methods for preparing whole blood RNA samples for microarray analysis. According to the present invention, a labelling protocol includes a step for blocking amplification of hemoglobin RNA at some point during the amplification process. Typically RNA amplification and labelling steps are carried out in a thermocycler using cellular RNA which has been purified from whole blood or other tissue samples. In one embodiment, (1) the oligonucleotides are preincubated with the total RNA prior to any amplification step. One specific example of this is provided in Example 2 below. (2) First strand cDNA is synthesized from pretreated RNA by incubating a quantity of RNA with a T7 promoter-dT-primer with reverse transcriptase (RT) and related reagents. These reagents and RT are incubated in a thermocyler. (3) Second strand cDNA synthesis is carried out using a DNA polymerase with the first strand cDNA as a template, along with RNAse H and DNA ligase. (4) In vitro transcription used labeled reagents such as biotin labeled ribonucleotides are used to generate labeled cRNA. This cRNA is purified, fragmented and used to hybridize to expression array chips containing large number of oligonucleotide probes. The patterns displayed on the arrays can be visualized and analyzed using commercially available bioanalyzers. FIG. 2 shows that the preincubation of whole blood RNA with the oligonucleotides of the present invention results in the reduction of labelled hemoglobin RNA.

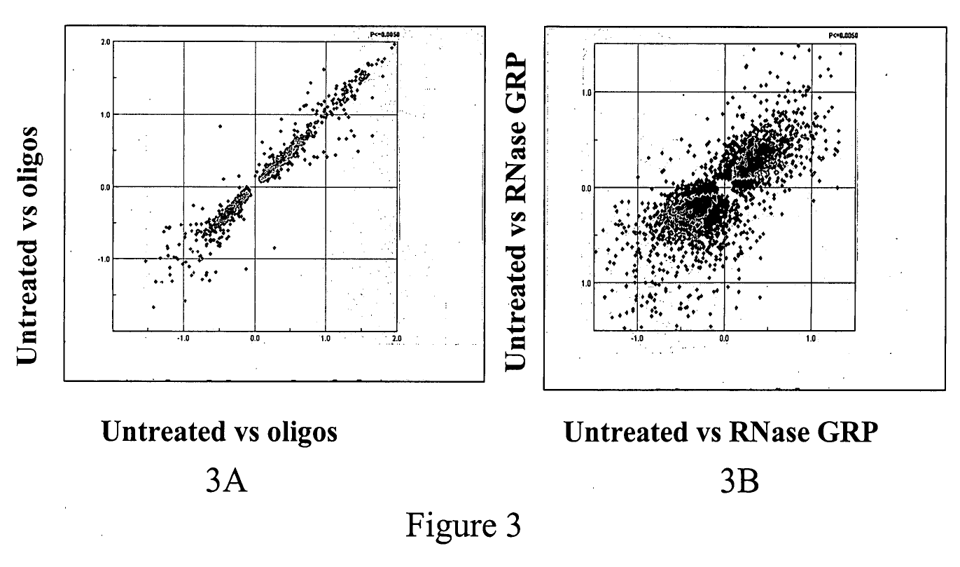

Use of the oligonucleotides of the present invention to prepare RNA samples for microarray analysis was compared to another method of hemoglobin reduction called the globin reduction method. The globin reduction protocol relies on the use of anti-hemoglobin conventional oligonucleotides hybridized to hemoglobin transcripts followed by RNAse H digestion and clean-up using column chromatography. This was following by labelling of the remaining RNA. In comparison to the globin reduction method, the LNA oligonucleotide method of the present invention provides more consistent results from sample to sample, and fewer off-target effects created by cross-hybridization of the oligos to unintended targets. This is shown in FIG. 3. FIG. 3 demonstrates that the oligonucleotides and methods of the present invention are particularly useful for preparing whole blood RNA samples for analysis on microarrays.

The invention having been described, the following examples are offered by way of illustration, and not limitation.

### EXAMPLE 1

### Preparation of Oligonucleotides

Six oligonucleotide sequences containing combinations of locked nucleotide analogs and standard deoxyribonucleotides were designed to hybridize with the 3′ ends of both the HBA1 mRNA transcript and the HBA2 mRNA transcript. The olignoculeotides which hybridize with the 3′ end of both HBA1 and HBA2 are the following: oligo #1, GCCCACtcacAGA (SEQ ID NO: 1); oligo #3, TTGccgcccACTC (SEQ ID NO: 3); oligo #5, TTGccgcccACTCA (SEQ ID NO: 5); and oligo #6, TTTAttcaaagaCCA (SEQ ID NO: 6). The capital letters refer to the nucleotide analogs containing a 2′-O, 4′-C-methylene bridge, while the small letters refer to the conventional nucleotides.

A second group of oligonucleotides was designed to hybridize with the HBB mRNA transcript. These oligonucleotides are oligo #2, CCCTTcataatatCCC (SEQ ID NO: 2), and oligo #4, CAAtgAAAAtAAATG (SEQ ID NO: 4).

These oligonucleotides were synthesized commercially by Proligo LLC (Boulder, Colo. 80301). Alternatively, the oligonucleotides can be prepared synthetically using commercially available LNA® phophoramidites from Proligo LLC, for example, according to manufacturer's instructions. Oligonucleotides can be prepared using standard phophoramidite synthesis protocols and commercially available solid supports such as is used for synthetic DNA oligomer synthesis with the modifications described in manufacturer's instructions. These modifications include a longer coupling time for the LNA monomers compared with conventional DNA monomers.

Each of the oligonucleotides was designed to have a desired Tm at hybridization conditions of 115 mM salt and 2 uM oligonucleotide concentration. The Tm of each of the specific oligonucleotides is:

| | | | |
|---|---|---|---|
| HBA oligo #1 | (GCCCACtcacAGA) | 78° C. | SEQ ID NO:1 |
| HBB oligo #2 | (CCCTTcataatatCCC) | 70° C. | SEQ ID NO:2 |
| HBA oligo #3 | (TTGccgcccACTC) | 78° C. | SEQ ID NO:3 |
| HBB oligo #4 | (CAAtgAAAAtAAATG) | 64° C. | SEQ ID NO:4 |
| HBA oligo #5 | (TTGccgcccACTCA) | 82° C. | SEQ ID NO:5 |
| HBA oligo #6 | (TTTAttcaaagaCCA) | 60° C. | SEQ ID NO:6 |

### Example 2

### Use of Oligonucleotides to Block Hemoglobin Transcript Amplification

The following protocol used oligos #2 and #3 to block HBA and HBB mRNA amplification in an RNA sample extracted from human whole blood.

Whole blood samples were collected from human subjects using PAXgene™ Blood RNA Tubes (PreAnalytiX, distributed in the US by Qiagen Inc. Valencia, Calif.). Cellular RNA was purified with DNase-digestion using the PAXgene™ Blood RNA Kit according to manufacturer's instructions. The quality of the total RNA was assessed by electrophoresis according to standard procedures. The RNA was checked for intactness and contained a ratio of 28S to 18S ribosomal RNA of around 2. Total RNA submitted was free of contaminating DNA. Optionally, the samples may be pretreated with RNAse before labelling.

5-10 ug of total RNA was used for each labelling reaction. The RNA sample was prepared for first strand cDNA synthesis using the following protocol.

1. The following reagents were added to an RNAse free 0.2 mL PCR tube:

2-10 ug total RNA

1 ug T7 promoter dT (24) primer (10 pmole/ul) (Invitrogen)

1 uL oligomer mix of 20 pmol/uL each of oligo #2 and oligo#3 water to 11 uL

Mixed using filter tips on the pipet.

2. The samples were incubated at 85° C. for five minutes in a thermocycler, then cooled to 70° C. at a rate of 0.1° C. per second in the thermocycler.

3. Cooled tubes on ice or at 4° C. in the thermocycler for 2-5 minutes.

4. Prepared a reverse transcription (RT) mastermix with the following components:

4 uL 5X First Strand Buffer (Invitrogen)

2 uL 100 mM DTT (Invitrogen, aliquoted to 100 uL per tube)

1 uL 10 mM dNTSP (Invitrogen)

Mixed briefly by vortex.

5. Added 7 uL of RT mastermix to each RNA/primer sample and mixed well by pipeting with filter tip.

6. Added 2 uL of Invitrogen Superscript Reverse Transcriptase enzyme (200 U/uL,Invitrogen). Total volume of reaction was 20 uL.

7. Mixed gently by flicking tube, and/or pipeted up and down 2-3 times slowly with a filter tip. Spined quickly to collect liquid in bottom of tube, if necessary.

8. Placed tubes in a thermocycler set for 42° C. and incubated for 1 hour, using heated lid setting. (preferably program to go to 4° C. at the end of the incubation.)

9. After 1 hour, placed tubes on ice or at 4° C. in thermocycler and prepared for second strand cDNA synthesis. Spinned in microfuge to collect any condensate which may have collected on the lid.

After treating the RNA sample with oligos #2 and #3 to block first strand cDNA synthesis of the HBA and HBB transcripts, the samples can be further processed. The first strand cDNA can be further amplified using PCR techniques known in the art. In one embodiment, the transcripts can be further processed to generate biotinylated cRNA for hybridization on Affymetrix GeneChip® Microarrays or other microarrays that are commercially available. The protocol provided by the vendor involves the further sequential steps of the production of second strand cDNA, cDNA purification, in vitro transcription (IVT) to generate biotinylated cRNA, IVT reaction mix purification, fragmentation of cRNA, preparation of hybridization mixtures, prehybridization of GeneChips® microarrays, and hybridization of cRNAs to GeneChips® microarrays.

Following the above protocol, samples of pretreated vs. nontreated RNA was compared for reduction of hemoglobin labelling. The results are shown in FIG. 2. It could be seen that a combination of oligos #2 and #3 effectively reduced the signal from hemoglobin in the RNA sample.

The pretreatment of RNA samples extracted from whole blood was also compared with identical samples treated with an Affymetrix Globin Reduction protocol. Globin reduction, according to the protocol used, involves the use of conventional DNA oligonucleotides complementary to the hemoglobin mRNA, followed by treatment of the sample with RnaseH, which cleaves RNA bound to DNA. The remaining mRNA is then processed and labeled for microchip analysis as described in the vendor's protocol.

A comparison of the two methods demonstrated that use of the oligonucleotides of the present invention results in more reproducible expression profiles, and a reduction in off-target labelling. Less off-target labelling results in more consistent results from sample to sample. FIG. 3 shows a comparison between the methods of the present invention compared to the globin reduction protocol.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

What is claimed is:

1. An oligonucleotide comprising at least one modified locked nucleotide analog, wherein the oligonucleotide is between about 8 to about 30 nucleotides in length, and wherein the oligonucleotide hybridizes to the 3′ end of hemoglobin A mRNA transcript or hemoglobin B mRNA transcript to form a heat stable duplex.

2. The oligonucleotide of claim 1, wherein the oligonucleotides comprises between about 5 and about 15 locked nucleotide analogs.

3. The oligonucleotide of claim 1, wherein the Tm of the oligonucleotide and the mRNA transcript duplex is between about 60° C. and about 82° C.

4. The oligonucleotide of claim 1, wherein the oligonucleotide sequence selected from the group consisting of:

| | | |
|---|---|---|
| (a) | GCCCACtcacAGA; | (SEQ ID NO:1) |
| (b) | CCCTTcataatatCCC; | (SEQ ID NO:2) |
| (c) | TTGccgcccACTC; | (SEQ ID NO:3) |
| (d) | CAAtgAAAAtAAATG; | (SEQ ID NO:4) |
| (e) | TTGccgcccACTCA, | (SEQ ID NO:5) |
| | and | |
| (f) | TTTAttcaaagaCCA. | (SEQ ID NO:6) |

wherein the capital letters refer to locked nucleotide analogs and the small letters refer to conventional nucleotides.

5. A composition comprising the oligonucleotides of claim 1.

6. A kit for use in blocking hemoglobin RNA amplification comprising the oligonucleotides of claim 1.

7. A kit for use in blocking hemoglobin RNA amplification comprising the oligonucleotides of claim 4.

8. A method of specifically blocking hemoglobin RNA amplification in a sample of RNA comprising contacting the sample with one or more of the oligonucleotides of claim 1.

9. The method of claim 8, wherein the RNA sample is pretreated with the oligonucleotides prior to contacting the sample with an amplification enzyme.

10. A method of specifically blocking hemoglobin RNA amplification in a sample of RNA comprising treating the sample with one or more of the oligonucleotides of claim 4.

11. The method of claim 10, wherein the RNA sample is pretreated with oligonucleotides prior to contacting the sample with an amplification enzyme.

12. The method of claim 10, wherein the RNA sample is treated with one or more of the oligonucleotides having SEQ ID NO: 1, 3, 5 and 6 in combination with one or more of the oligonucleotides having SEQ ID NO: 2 or 4.

13. The method of claim 10, wherein the concentration of oligonucleotide is between about 2 uM and about 5 uM oligonucleotide.

14. The method of claim 8, further comprising labelling the RNA sample.

15. The method of claim 14, further comprising analyzing the labelled RNA using microarray analysis.

16. The method of claim 10, further comprising labelling the RNA sample.

17. The method of claim 16, further comprising analyzing the labelled RNA using microarray analysis.


SEQUENCE LISTING (9 sequences)

SEQ ID NO: 1 - DNA, 13 bp, Artificial Sequence
  modified oligonucleotide
gcccactcacaga

SEQ ID NO: 2 - DNA, 16 bp, Artificial Sequence
  modified oligonucleotide
cccttcataatatccc

SEQ ID NO: 3 - DNA, 13 bp, Artificial Sequence
  modified oligonucleotide
ttgccgcccactc

SEQ ID NO: 4 - DNA, 15 bp, Artificial Sequence
  modified oligonucleotide
caatgaaaataaatg

SEQ ID NO: 5 - DNA, 14 bp, Artificial Sequence
  modified oligonucleotide
ttgccgcccactca

SEQ ID NO: 6 - DNA, 15 bp, Artificial Sequence
  modified oligonucleotide
tttattcaaagacca

SEQ ID NO: 7 - DNA, 576 bp, Homo sapiens
actcttctggtccccacagactcagagagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtggccatgcttcttgccccttgggcctccccccagcccctcctccccttcctgcacccgtacccccgtggtctttgaataaagtctgagtgggcggc

SEQ ID NO: 8 - DNA, 575 bp, Homo sapiens
actcttctggtccccacagactcagagagaacccaccatggtgctgtctcctgccgacaagaccaacgtcaaggccgcctggggtaaggtcggcgcgcacgctggcgagtatggtgcggaggccctggagaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtagccgttcctcctgcccgctgggcctcccaacgggccctcctcccctccttgcaccggcccttcctggtctttgaataaagtctgagtgggcggc

SEQ ID NO: 9 - DNA, 626 bp, Homo sapiens
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgc